(19) United States
(12) Patent Application Publication
(10) Pub. No.: US 2002/0183388 A1
(43) Pub. Date: Dec. 5, 2002

(54) Use of retinoids plus histone deacetylase inhibitors to inhibit the growth of solid tumors

(76) Inventors: Lorraine J. Gudas, New York, NY (US); David Nanus, New Rochelle, NY (US)

Correspondence Address: Michael L. Goldman, Esq., NIXON PEABODY LLP, Clinton Square, P.O. Box 31051, Rochester, NY, 14603-1051, US

(21) Appl. No.: 10/061,101

(22) Filed: Feb. 1, 2002

Related U.S. Application Data
(60) Provisional application No. 60/265,651, filed on Feb. 1, 2001.

(51) Int. Cl. A61K 31/203; A61K 31/19; A61K 31/16

(52) U.S. Cl. 514/559; 514/557; 514/575

(57) ABSTRACT

The present invention provides a method of inhibiting growth of solid tumors in an animal which comprises administering an effective amount of trichostatin A (TSA) to an animal in need of such treatment. The present invention also provides a method of inhibiting growth of solid tumors in an animal which comprises administering an effective amount of a histone deacetylase inhibitor and a retinoid to an animal in need of such treatment. Examples of solid tumors which may be treated using the methods of the invention include but are not limited to carcinomas of the head and neck, breast, skin, kidney, oral cavity, colon, prostate, pancreas and lung.

This application claims priority from U.S. Provisional Application Serial No. 60/265,651, filed Feb. 1, 2001, which is hereby incorporated by reference.

This invention was made with government support under Grant numbers R01DE10389 and R01CA77509 by the National Institutes of Health. The government has certain rights in the invention.

### BACKGROUND OF THE INVENTION

Tumors are generally classified as either solid or liquid (hematopoietic). Examples of solid tumors include carcinomas of the head and neck, breast, skin, kidney, prostate, colon, pancreas and lung. Liquid tumors include for example, leukemia and lymphoma. In the clinical setting, tumors are generally treated differently based on whether they are solid or liquid. Solid tumors are generally treated via surgical methods or a combination of surgical methods and radiation therapy. If metastasis is observed, drug therapy may be further employed. In the cases of a liquid tumor, drug therapy is the most often used course of treatment. The present invention provides methods for treatment of solid tumors.

### SUMMARY OF THE INVENTION

The present invention provides a method of inhibiting growth of solid tumors in an animal which comprises administering an effective amount of trichostatin A (TSA) to an animal in need of such treatment. Preferably, the animal is a mammal. Even more preferably, the mammal is a human.

In accordance with the present invention there is also provided a method of inhibiting growth of solid tumors in an animal which comprises administering an effective amount of a histone deacetylase inhibitor and a retinoid to an animal in need of such treatment.

Examples of solid tumors which may be treated using the methods of the invention include but are not limited to carcinomas of the head and neck, breast, skin, kidney, oral cavity, colon, prostate, pancreas or lung.

In accordance with the present invention, a histone deacetylase inhibitor and retinoid may be administered serially or in combination.

Examples of histone deacetylase inhibitors which may be used in the methods of the present invention include but are not limited to Trichostatin A, Trichostatin C, butyric acid, potassium butyrate, sodium butyrate, ammonium butyrate, lithium butyrate, phenylbutyrate, sodium phenylbutyrate (NaPBA), a stable butyrate derivative, traponin, valproic acid or SAHA.

Examples of retinoids which may be used in accordance with the present invention include but are not limited to retinol, 9-cis retinoic acid, 13-cis retinoic acid, all-trans retinoic acid, 4-oxoretinol, 4-oxoretinaldehyde or a retinyl ester.

If desired, the retinoid used in the methods of the present invention may be in a lipid formulation such as liposomal ATRA Tretinoin.

### BRIEF DESCRIPTION OF THE DRAWINGS

FIG. 1 graphically depicts results of growth assays of 5 and 7 day MB-435 cells treated as indicated. Assay was performed by MTT.

FIG. 2 graphically depicts results of growth assays of HMEC cells treated as indicated. Cell number was counted.

FIG. 3 graphically depicts results of growth assays of 3, 5, and 7 day MB-435 cells treated as indicated. Assay was performed by MTT.

FIG. 4 graphically depicts results of growth assays of 3, 5, and 7 day MB-231 cells treated as indicated. Assay was performed by MTT.

FIG. 5 graphically depicts results of growth assays of 3, 5, and 7 day MCF-7 cells treated as indicated. Assay was performed by MTT.

FIG. 6 graphically depicts results of growth assays of SK-RC-39 cells treated as indicated. Cell number was counted.

FIG. 7 graphically depicts results of growth assays of SK-RC-01 cells treated as indicated. Cell number was counted.

FIG. 8 graphically depicts results of growth assays of SK-RC-45 cells treated as indicated. Cell number was counted.

FIG. 9 graphically depicts results of growth assays of SK-RC-39 cells treated as indicated. Cell number was counted.

FIG. 10 graphically depicts results of growth assays of SK-RC-39 cells treated as indicated. Cell number was counted.

FIG. 11 graphically depicts results of growth assays of SK-RC-39 cells treated as indicated. Cell number was counted.

FIG. 12 graphically depicts results of growth assays of SK-RC-01 cells treated as indicated. Cell number was counted.

FIG. 13 graphically depicts results of growth assays of SK-RC-45 cells treated as indicated. Cell number was counted.

FIG. 14 graphically depicts results of growth assays of SK-RC-45 cells treated as indicated. Cell number was counted.

FIG. 15 is a growth curve for SK-RC-39 cells treated as indicated. Cell number was counted.

FIG. 16 is data from a human tumor xenograft in nu/nu mice. Mice were treated as indicated in Example 2.

FIG. 17 graphically depicts the results of growth assays of LnCap cells treated as indicated. Cell number was counted.

FIG. 18 depicts results of growth assays of SCC-15 cells treated as indicated. Cell number was counted.

FIG. 19 graphically depicts results of growth assays of SCC-15 cells treated as indicated. Cell number was counted.

FIG. 20 graphically depicts results of growth assays of PC-3 cells treated as indicated. Cell number was counted.

### DETAILED DESCRIPTION OF THE INVENTION

In accordance with the present invention, it has been surprisingly found that administration of trichostatin A is effective in the treatment of solid tumors in vivo. It has also been surprisingly found that administration of both a histone deacetylase (HDAC) inhibitor and a retinoid (synthetic derivatives of vitamin A, retinol), is effective in the treatment of solid tumors in vivo.

Thus, in accordance with the present invention, there is provided a method of inhibiting the growth of solid tumors in vivo by administering to a patient an effective amount of trichostatin A. Also in accordance with the present invention, there is provided a method of inhibiting the growth of solid tumors in vivo by administering to a patient an effective amount of a histone deacetylase (HDAC) inhibitor and a retinoid. The administration of a histone deacetylase (HDAC) inhibitor and a retinoid may be performed in serial or in combination. When administered in combination, the HDAC and retinoid may comprise a mixture which is administered to the patient. Alternatively, when administered in combination, the HDAC and retinoid may be administered separately but simultaneously as in for example, two separate i.v. lines.

Examples of solid tumors which may be treated in accordance with the methods of the present invention include for example, carcinomas of the head and neck, breast, skin, kidney, oral cavity, colon, prostate, pancreas, and lung.

Examples of histone deacetylase inhibitors which may be used in accordance with the methods of the present invention include for example, Trichostatin A, Trichostatin C, butyric acid and butyric acid salts such as potassium butyrate, sodium butyrate, ammonium butyrate, lithium butyrate, phenylbutyrate, and sodium phenylbutyrate (NaPBA); stable butyrate derivatives, traponin, valproic acid, suberoylanilide hydroxamic acid (SAHA), etc.

Examples of retinoids which may be used in the methods of the present invention include e.g., retinol, 9-cis retinoic acid, 13-cis retinoic acid, all-trans retinoic acid ( also referred to as tretinoin, all-trans RA or ATRA), 4-oxoretinol, 4-oxoretinaldehyde and retinyl esters. If desired, liposomal preparations of retinoids may be used in the methods of the present invention. For example, liposomal tretinoins such as liposomal ATRA Tretinoin or ATRA-IV may be used. A liposomal delivery system improves the activity of tretinoin by altering its pharmacological profile, changing the drug's pharmacokinetics and tissue distribution. In vitro, liposomal ATRA has a greater antiproliferative effect on neoplastic cells than free-ATRA. In vivo, liposomes bypass the clearance mechanism that evolves in the livers of patients treated with the oral formulation. In addition, toxicities associated with oral doses of tretinoin might be reduced because liposome encapsulation of tretinoin decreases direct exposure of the tretinoin during circulation to levels below the orally administered toxic dose. The latter allows greater total exposure of the drug on initial dose accompanied by slower clearance of the tretinoin.

The histone deacetylase inhibitors and retinoids for use in the methods of the present invention may be prepared for convenient and effective administration in pharmaceutically effective amounts with a suitable pharmaceutically acceptable carrier. Pharmaceutical acceptable carriers may include for example, solvents, dispersion media, antibacterial and antifungal agents, microcapsules, liposomes, cationic lipid carriers, isotonic and absorption delaying agents and the like which are not incompatible with the active ingredients. The formulation of pharmaceutical compositions is generally known in the art and reference may be conveniently made to Remington's Pharmaceutical Sciences, 17^{th} ed., Mack Publishing Co., Easton, Pa. The active ingredients of a pharmaceutical composition comprising a retinoid and/or histone deacetylase inhibitor are contemplated to exhibit excellent therapeutic activity for treating a variety of solid tumors, when administered in an amount which depends on the particular case.

Thus, an effective amount of a histone deacetylase inhibitor or an effective amount of a retinoid and a histone deacetylase inhibitor is administered to a patient suffering from a solid tumor(s). By “effective amount” is meant an amount effective to inhibit the growth of the tumor(s) in vivo.

ATRA-IV is a lipid formulation of tretinoin (USP) suitable for intravenous infusion. Common synonyms for tretinoin are: RA, all-trans-retinoic acid, vitamin A acid, or 3, 7 dimethyl-9-(2,6,6-trimethyl-1-cyclohenen-1-yl)-2, 4, 6, 8,-nonatetraenoic acid [CAS No. 302-79-4]. ATRA-IV is a lyophilized mixture of 935 mg dimyristoyl phosphatidyl choline (or DMPC), 165 mg soybean oil, USP and 110 mg tretinoin, USP. A vial of product appears as yellow lyophilized powder. The lyophilized powder is reconstituted at point of use with 0.9% sodium chloride for injection, USP to form a liposome suspension. The reconstituted suspension contains 2 mg/ml of tretinoin. A vial of lyophilized ATRA-IV is reconstituted with 50 ml of 0.9% sodium chloride for injection, USP, to provide a 2 mg per ml of liposomal suspension requiring no further dilution steps.

In accordance with the methods of the present invention, a histone deacetylase inhibitor such as TSA is administered in a manner compatible with the dosage formulation and in such amount as will be therapeutically effective, i.e., an amount effective to inhibit growth of a solid tumor. Also in accordance with the methods of the present invention, a retinoid such as an all-trans retinoic acid is administered in a manner compatible with the dosage formulation and in such amount as to be therapeutically effective, i.e., an amount effective to inhibit growth of a solid tumor. Systemic dosages depend on the age, weight, condition of the patient, size of tumor(s), and administration route.

A histone deacetylase inhibitor and/or retinoid may be administered in any way which is medically acceptable. Possible administration routes include intravascular, intravenous, intraarterial, subcutaneous, intramuscular, intratumor, intraperitoneal, intraventricular, intraepidural, or others.

For example, ATRA-IV may be administered to a subject via intra-arterial or intravenous infusion in a dosage range of from about 15 to about 75 mg/m².

In accordance with the methods of the present invention, a histone deacetylase inhibitor such as sodium phenylbutyrate (NaPBA) may be administered in a dosage of anywhere in the range of from about 9.9 to about 13 g/m² orally, divided three times daily. Alternatively, a dosage of about 19 g/d, orally, for one week may be administered. Other dosage regimes include for example, a dose range of from about 150 mg/kg IV every other day in increments of about 50 mg/kg until maximum tolerated dose (MTD) is reached or until about 400 mg/kg about 28 gm/d in the average male).

Oral formulations may include for example, an inert diluent, an assimilable edible carrier and the like, be in hard or soft shell gelatin capsule, be compressed into tablets, or may be in an elixir, suspension, syrup, or the like.

Dosage regima may be adjusted to provide the optimum therapeutic response. For example, several divided doses may be administered daily or the dose may be proportionally reduced or increased as indicated by the specific therapeutic situation.

In accordance with the present invention, “subject” is meant to include any animal including birds. In a preferred embodiment of the invention, the animal is a mammal. In a most preferred embodiment of the invention, the mammal is a human.

The invention is further illustrated by the following specific examples which are not intended in any way to limit the scope of the invention.

### EXAMPLE 1

MALIGNANT CELL GROWTH ASSAYS

| Materials and Methods: | |
|---|---|
| The following cultured, human tumor lines were used: | |
| MB-435 | human breast cancer |
| MB-231 | human breast cancer |
| MCF-7 | human breast cancer |
| SK-RC-39 | human renal carcinoma |
| SK-RC-01 | human renal carcinoma |
| SK-RC-45 | human renal carcinoma |
| SCC-15 | human head and neck cancer |
| LnCap | human prostate cancer |
| PC-3 | human prostate cancer |

Also used were HMEC cells, which are cells from a normal human mammary epithelial cell line (primary cultures).

Drugs Used and Cell Cultures:

Liposome encapsulated ATRA (ATRA-IV, Antigenics Inc., New York, N.Y.) was reconstituted from 100 mg vials with 50 ml of 0.9% saline. Powdered ATRA was dissolved in EtOH to make a 1 mM ATRA (Sigma, St. Louis, Mo.) stock which was stored at −80° C. Trichostatin A (Wako Pure Chemical Industries, Ltd., Osaka, Japan) was dissolved in 5 ml of EtOH to make a 1 mg/ml stock and sodium phenylbutyrate (NaPBA, Triple Crown America, Perkasie, Pa.) was prepared weekly from the powdered form to make a 25 mM stock solution. Cell lines were maintained in Minimal Essential Medium supplemented with 7% fetal calf serum, non-essential amino acids, and 1% penicillin/streptomycin (7% MEM). Empty liposomes (provided by Antigenics Inc.) were diluted in 10 ml of 0.9% saline to equal the same concentration of liposomes in the lipoATRA. SK-RC-01, SK-RC-39 and SK-RC-45 RCC cell lines were derived as described previously (Ebert et al. 1990, “Establishment and characterization of human renal cancer and normal kidney cell lines,” Cancer Res., 50:5531-5536).

96 well tissue culture dishes were seeded with 10⁴ cells/well in 200 μL DME tissue culture medium supplemented with 10% fetal calf serum (FCS). Cells were incubated overnight at 37° C. in 0% CO₂. Fresh medium containing drugs: Ethanol control (EtOH), 1 μM retinoic acid (RA), 1 μM ATRA-IV, 2 ng/mL trichostatin A (TSA), 500 μM sodium phenylbutyrate (PBa), and combinations of these were added the next day (day zero). Fresh medium containing drugs was added on days 3 and 5.

MTT assay:

This assay detects color which is proportional to the number of cells present. Tissue culture medium containing drugs was removed and 200 L of DME containing 2 mg/mL MTT ((3-4, 5-dimethyl thiazol-2yl) 2,5-diphenyltetrazolium bromide) was added to each well. Plates were incubated at 37° C. and 10% CO₂ for 30 minutes. Medium containing MTT was then removed and the cells were washed once with PBS. Following removal of the PBS, 200 μL DMSO was added to each well and plates were placed on a rotating platform shaker for 5 minutes. Results were obtained by reading the absorbance at a wavelength of 550 nm using a spectrophotometer. Data was plotted using Prism 3.0. Error bars are the standard error mean (SEM) of 4 replicate wells.

Growth Curves:

24 well tissue culture dishes were seeded with cells in 1 mL tissue culture medium (Clonetics) at 10⁴ cells/well. Cells were incubated overnight at 37° C. in 10% CO₂. Fresh medium containing drugs: Ethanol control (EtOH), 1 μM retinoic acid (RA), 2ng/mL trichostatin A (TSA), 25 μM sodium phenylbutyrate (PBa), and combinations of these were added the next day (day zero). Fresh medium containing drugs was added on days 3 and 5. Cells were trypsinized on days 0, 3, 5, and 7 and cell number was determined using a Z1 Coulter particle counter. Data was plotted using Prism 3.0. Error bars are the standard error mean (SEM) of 4 replicate wells.

Results

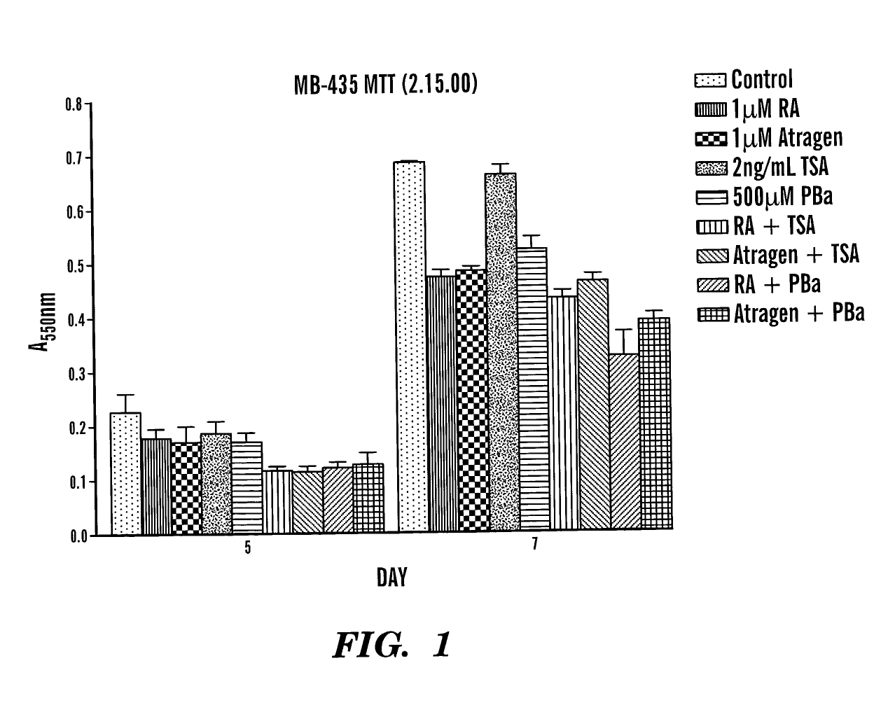

FIG. 1 graphically depicts the results of growth assays at 5 and 7 days of the human breast cancer cell line MB-435, treated as indicated. Cells were plated in wells on day 0. Drugs were added on day 0 and again on days 3 and 5. The MTT assay was carried out on days 5 and 7. 1 μM of retinoic acid was used vs. 1 μM of liposomal retinoic acid (ATRA-IV). A very low dose (2 ng/ml) of trichostatin A (TSA) was also used. In addition, another inhibitor of histone deacetylases, 500 μM phenylbutyrate, was used. Various combinations of the drugs were also used in the growth assays. As can be seen in FIG. 1, retinoic acid and ATRA-IV inhibited cell growth by about 20% on day 7, while low dose TSA did not inhibit cell growth. Phenylbutyrate alone inhibited cell growth by 15-20%. The combinations of retinoids plus histone deacetylase inhibitors were more effective at inhibiting cell growth as measured by the MTT assay.

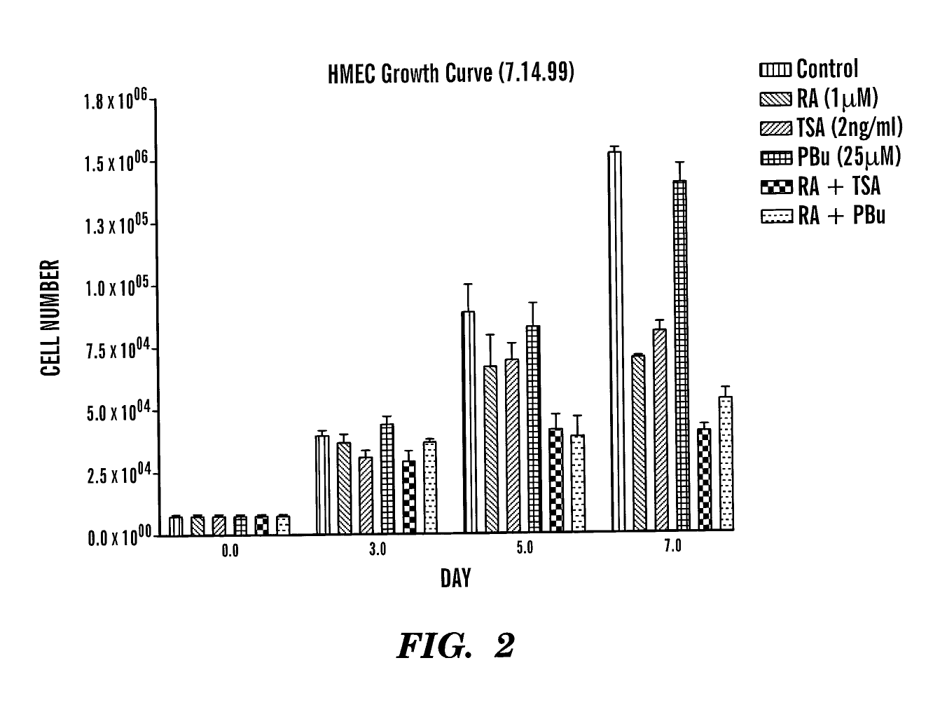

FIG. 2 graphically depicts the results of growth assays of normal human mammary epithelial cells (primary cultures) treated as indicated. The drugs were added as described above, and cells were counted on days 0, 3, 5, and 7. In these cells, phenylbutyrate at 25 μM alone did not inhibit cell growth significantly. Retinoic acid at 1 μM inhibited cell growth by about 50%, as did the low dose (2 ng/ml) TSA. The combination of retinoic acid plus TSA or retinoic acid plus phenylbutyrate inhibited cell growth by about 75-80%. Thus, the combinations were more effective than each of the drugs alone. This figure shows that when normal human mammary epithelial cells are cultured in the presence of these drugs and are rapidly dividing in this cell culture system, there is some inhibition of cell growth. However, in the body, most normal cells are not rapidly dividing as tumor cells are. Thus, while this result indicates that retinoids and TSA can inhibit the growth and induce the differentiation of normal epithelial cells, these assays are not likely to represent fully the behavior of normal epithelial cells within the body. This experiment was primarily performed in order to determine if the retinoid and histone deacetylase inhibitor combination worked similarly in tumor and normal epithelial cells.

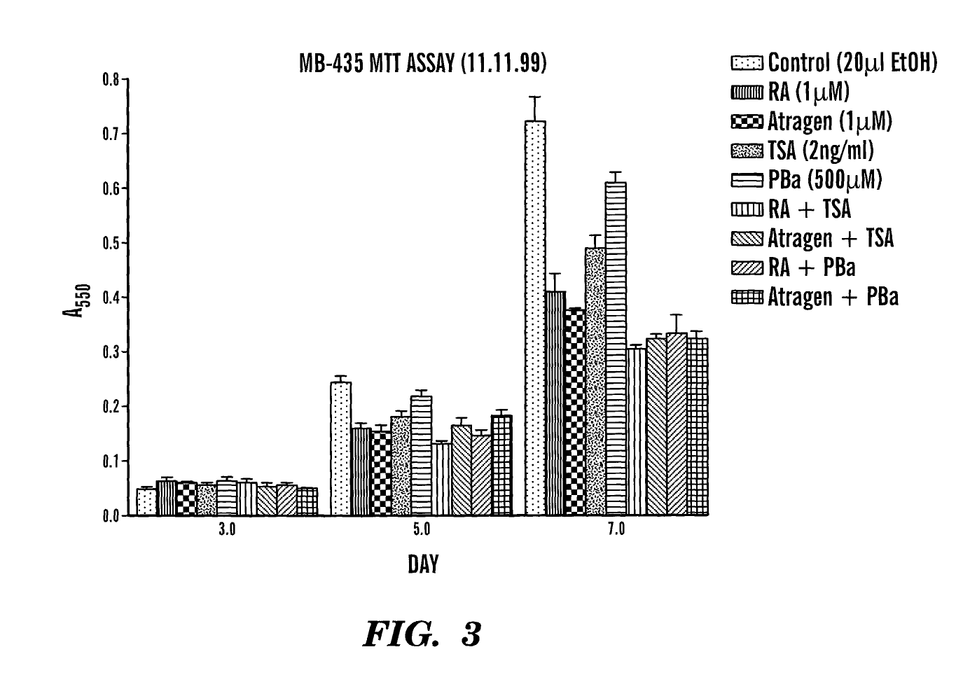

Results of growth assays of 3, 5, and 7 day human breast cancer MB-435 cells, measured by the MTT assay, are depicted in FIG. 3. Cells were treated as indicated. Results demonstrate that the combinations of retinoids plus histone deacetylase inhibitors were slightly more effective than either retinoic acid or ATRA-IV at inhibiting cell growth. TSA alone was better in reducing cell growth than phenylbutyrate. Retinoic acid and TSA reduced cell growth the greatest amount. This may be the case because phenylbutyrate is not as potent or specific an inhibitor of histone deacetylases as TSA. Phenylbutyrate can also be metabolized by cells to different extents. Thus, it is possible that in this particular cell line, phenylbutyrate was metabolized so that less of this histone deacetylase inhibitor was active in the cells as compared to TSA at a much lower dose. The combination of retinoic acid plus TSA resulted in a 50% inhibition of cell growth. A high dose of TSA (100 ng/ml) resulted in complete growth inhibition.

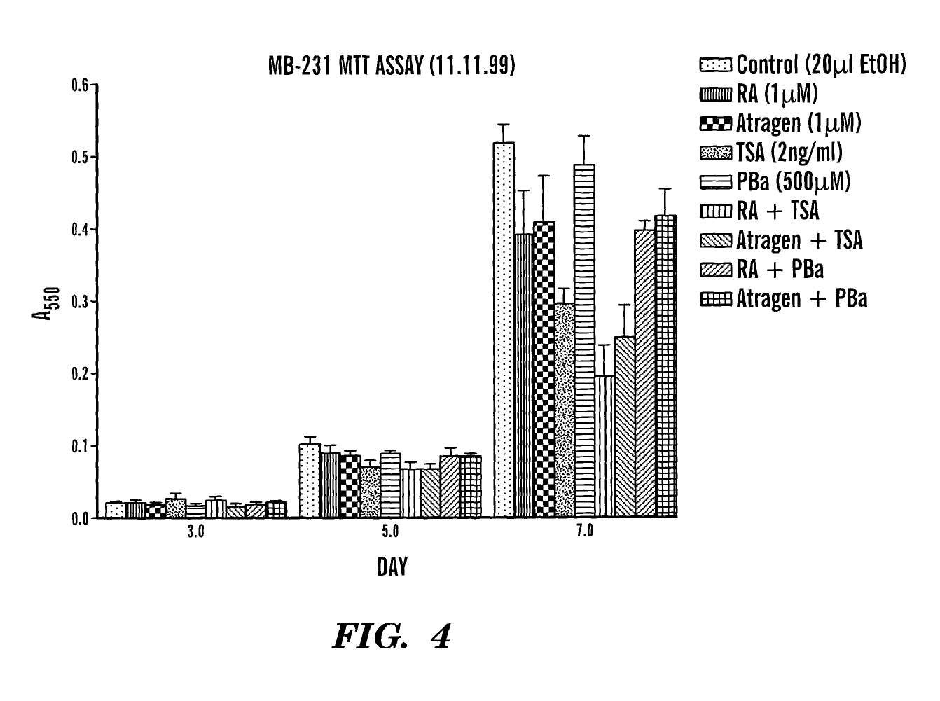

FIG. 4 graphically depicts the results of growth assays of 3, 5, and 7 day MB-231 human breast cancer cells treated as indicated. The growth was measured by the MTT assay. On these cells, the combinations of ATRA-IV plus TSA and retinoic acid plus TSA were much more effective at inhibiting cell growth than each of these compounds alone. Results also indicated that the combination of retinoids plus phenylbutyrate was not more effective than the retinoids alone in inhibiting growth.

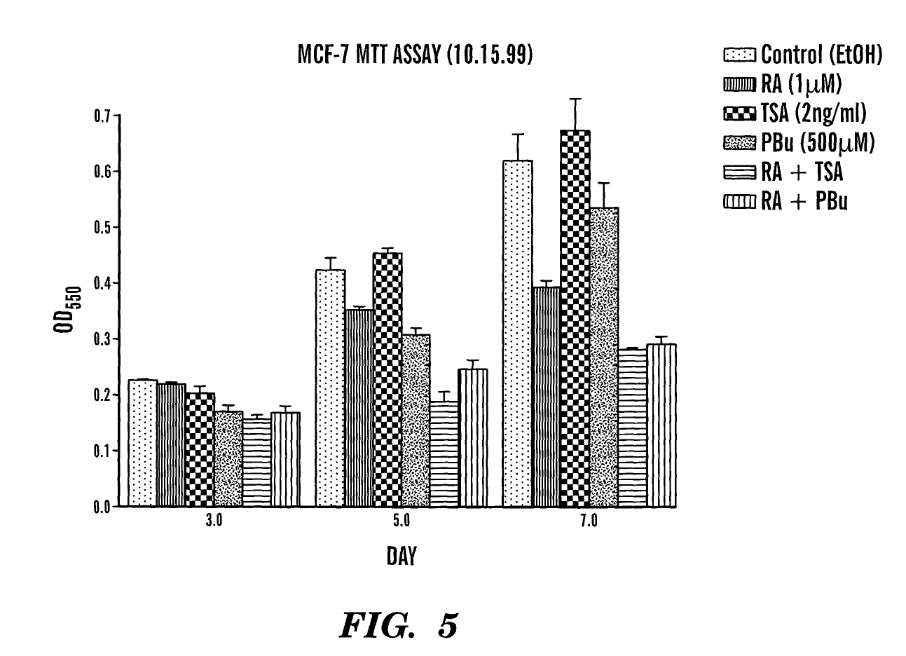

Results of growth assays of 3, 5, and 7 day MCF-7 human breast cancer cells are depicted in FIG. 5. Cells were treated as indicated. The growth was measured by MTT assay. On day 7, it can be seen that the combinations of retinoic acid plus low dose TSA and retinoic acid plus phenylbutyrate were significantly more growth inhibitory than each of these compounds given alone to the cells.

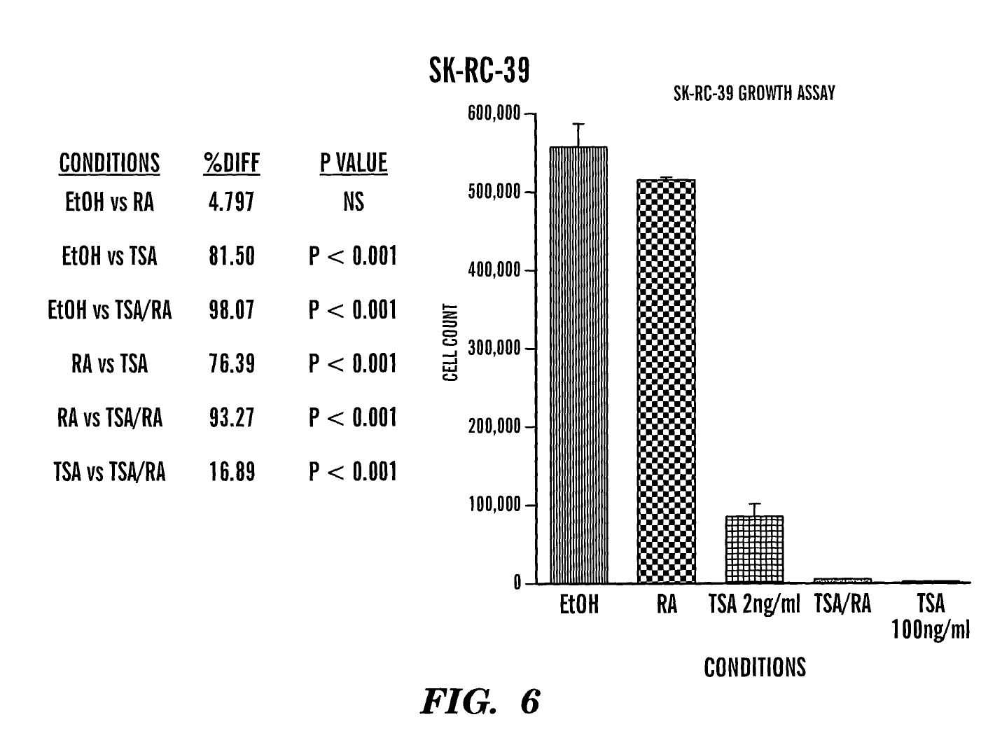

FIG. 6 graphically depicts the results of growth assays of human kidney cancer cells, SK-RC-39, treated as indicated. The cells were counted at day 7. A small amount of ethanol was added to the control cells, as a vehicle and solvent for the retinoic acid. In this experiment, retinoic acid alone had little growth inhibitory activity, while low dose TSA inhibited cell growth substantially (80-90%). The combination of retinoic acid plus low dose TSA completely inhibited cell growth (roughly 100% inhibition of growth).

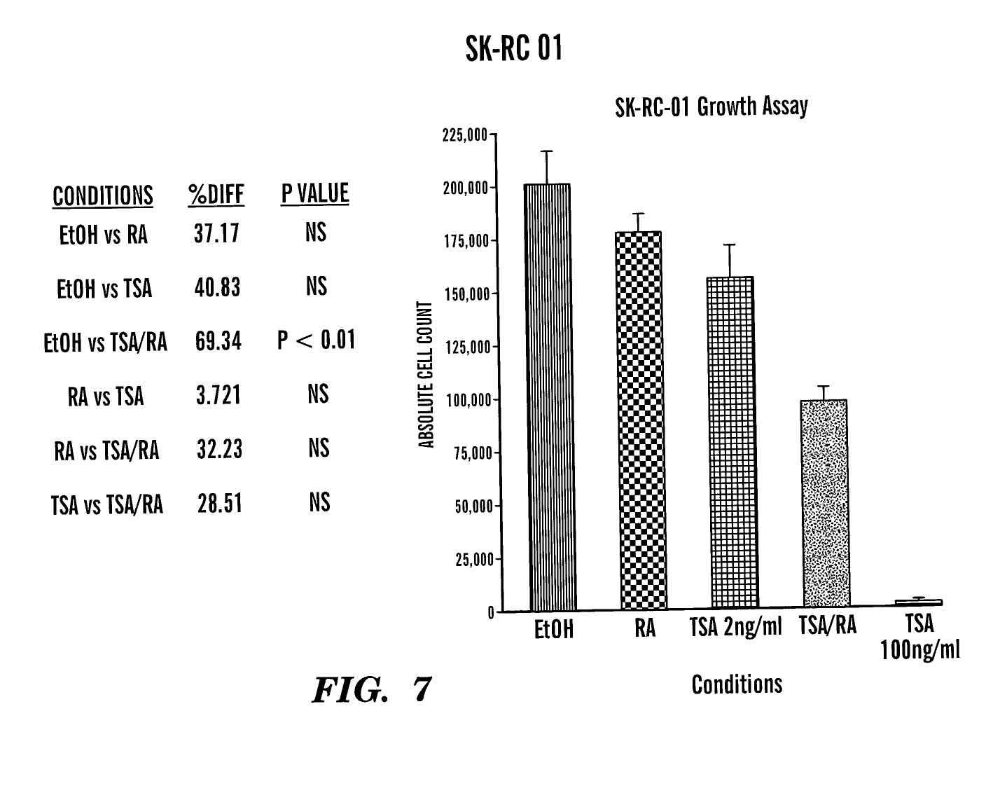

Results of growth assays of SK-RC-01 human kidney cancer cells are depicted in FIG. 7. Cells were treated as indicated. The cell number was counted at day 7. In this human kidney cancer line, retinoic acid alone resulted in about a 10% inhibition of cell growth. Low dose TSA alone resulted in a 20-25% inhibition of cell growth. The combination of retinoic acid plus low dose TSA resulted in a 50% inhibition of cell growth. A high dose of TSA (100 ng/ml) resulted in complete growth inhibition (roughly 100% inhibition).

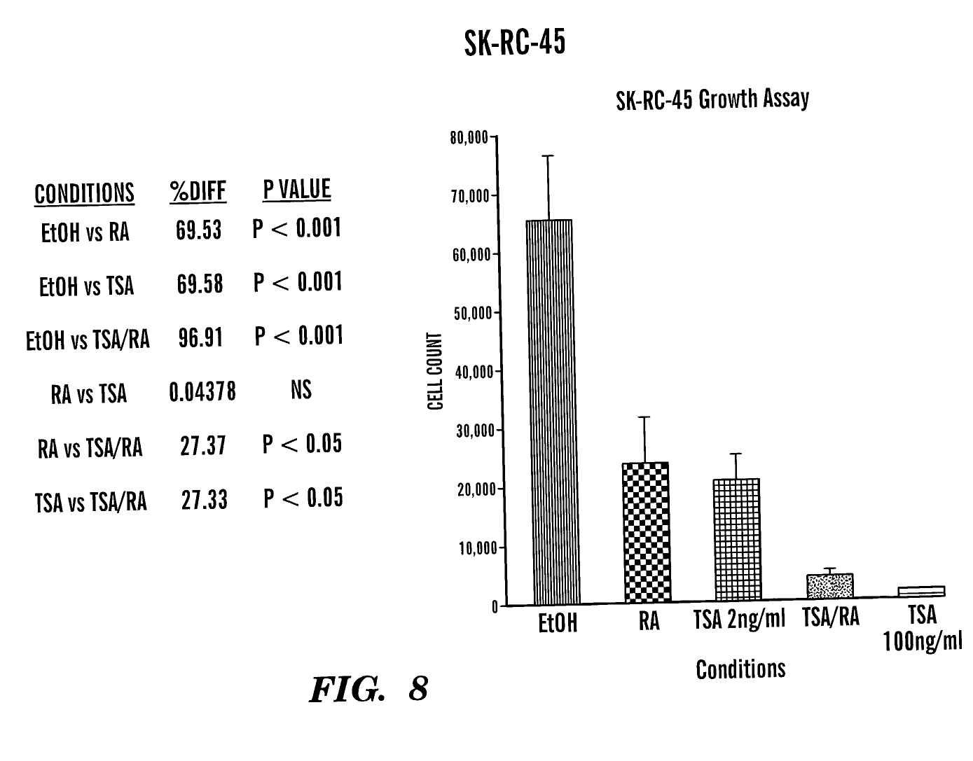

Results of growth assays of the human kidney cancer cell line SK-RC-45, are shown in FIG. 8. Cells were treated as indicated. The cell number was counted on day 7. In this experiment, 1 μM retinoic acid alone inhibited the growth of the cells by about 55-60%. Low dose TSA also inhibited the growth of the cells by approximately 60-65%. The combination of low dose TSA and retinoic acid inhibited cell growth by over 90%, and high dose TSA (100 ng/ml) was also extremely effective in inhibiting kidney cancer cell growth.

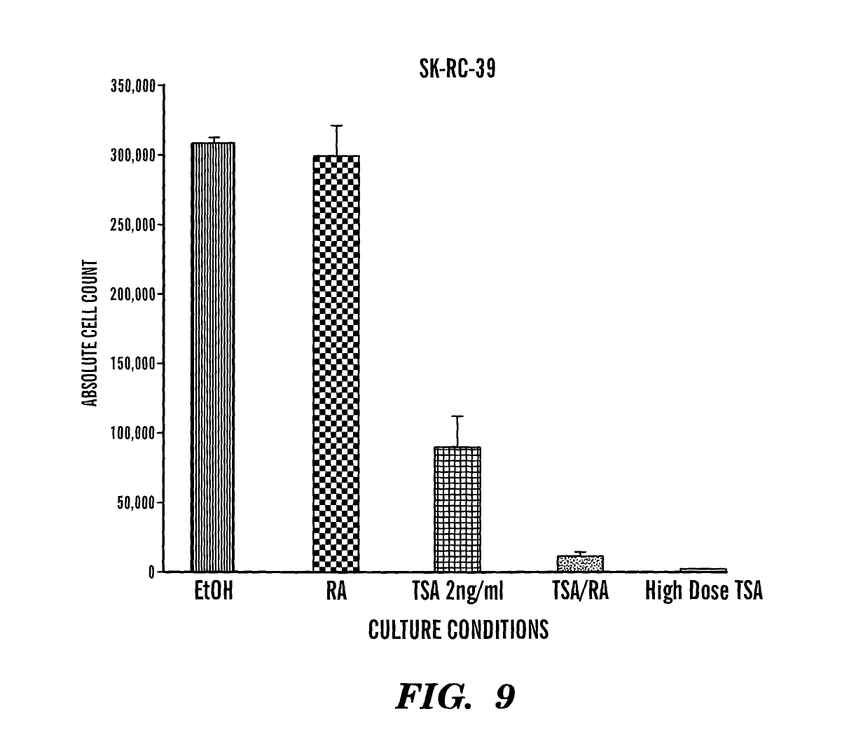

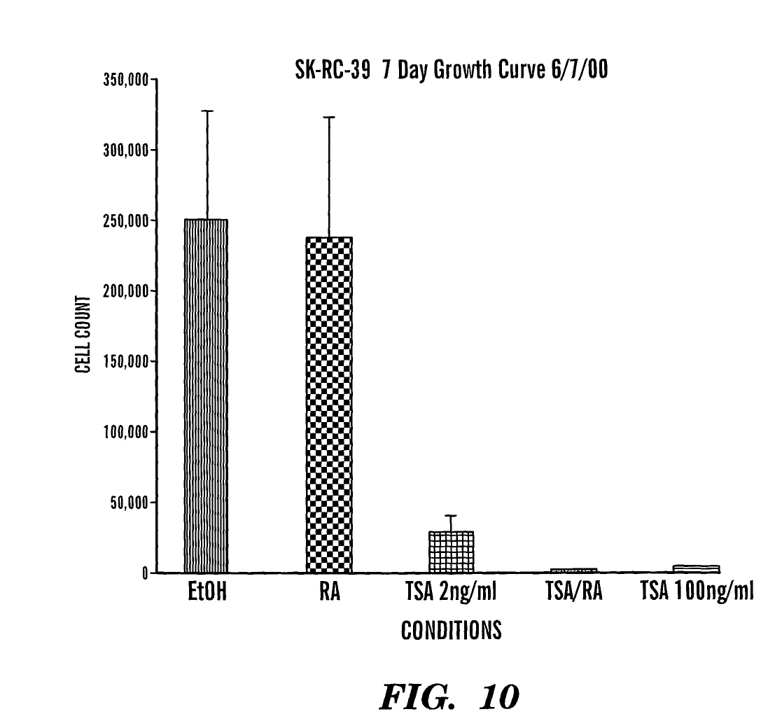

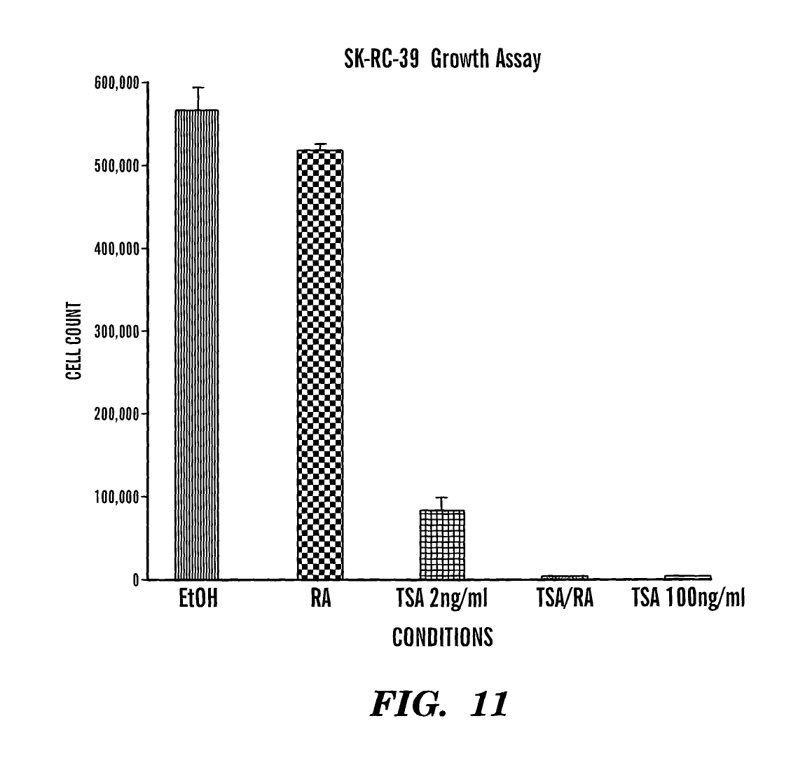

Results of growth assays of the human kidney cancer cells, SK-RC-39, are illustrated in FIG. 9. Culture conditions were as shown. The cell number was counted on day 7. The term EtOH indicates the control cells, treated with a very low amount of ethanol, the same amount used to dissolve retinoic acid. In this experiment, 1 μM retinoic acid alone did not inhibit cell growth, whereas low dose TSA inhibited cell growth by approximately 75%. The combination of low dose TSA and 1 μM retinoic acid inhibited cell growth by greater than 90%, as did high dose TSA. FIGS. 10 and 11 illustrate similar results using the same cell line under different treatments as indicated.

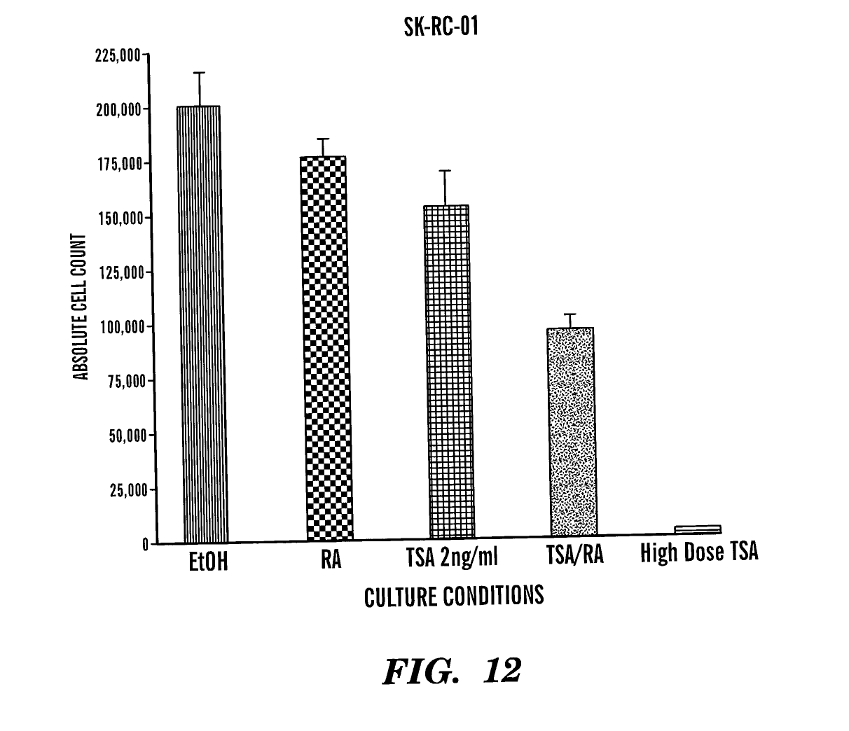

Results of growth assays of the human kidney cancer cells, SK-RC-01 are graphically depicted in FIG. 12. The cell number was counted on day 7. This graph shows that retinoic acid inhibited cell growth by 12.5%, low dose TSA by approximately 25%, and the combination by 50%. The high dose TSA inhibited cells by greater than 95%. Thus, 1 μM retinoic acid was effective in reducing cell growth, but was improved considerably when combined with TSA.

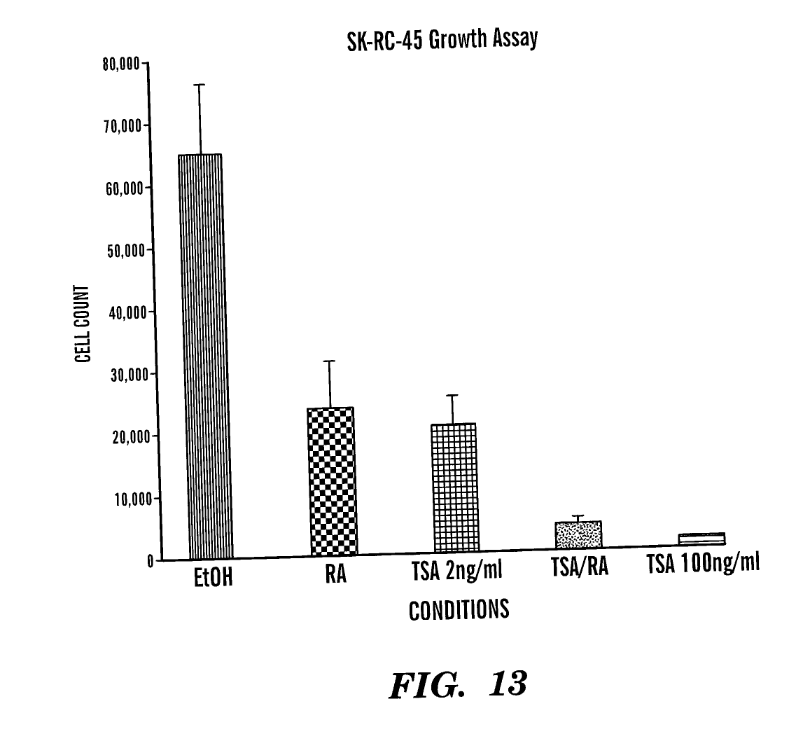

FIG. 13 graphically depicts the results of growth assays of the human kidney cancer line SK-RC-45, treated as indicated. Cell number was counted. The combination of retinoic acid and TSA was much more effective than either compound alone.

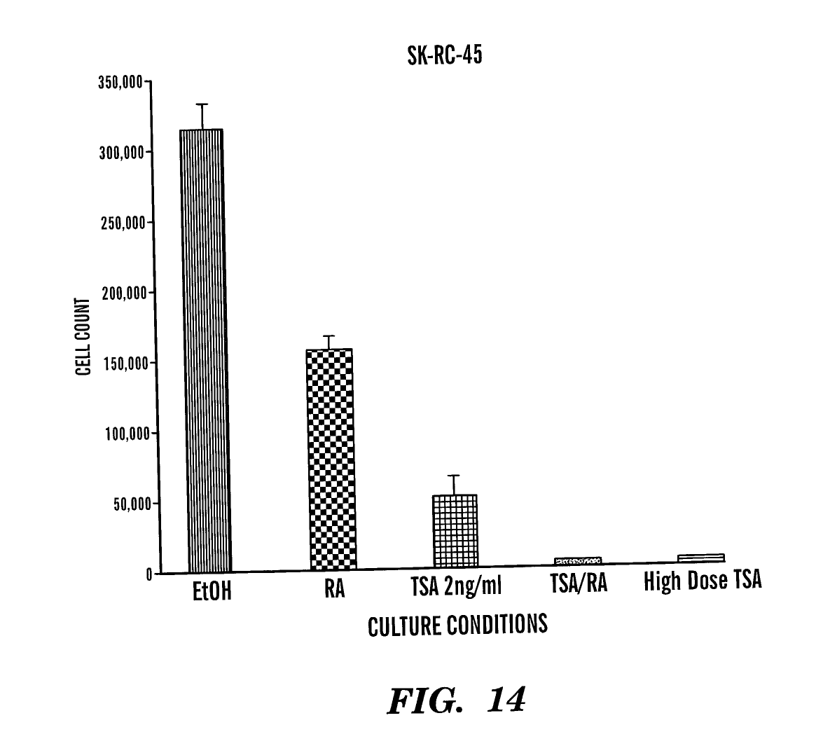

In FIG. 14, the results of growth assays of SK-RC-45 cells are shown. Cells were treated as indicated and cell number was counted. The results are similar to those in FIG. 13 and show that the combination of retinoic acid plus TSA was much more effective than either drug at the same dose administered to the cells alone.

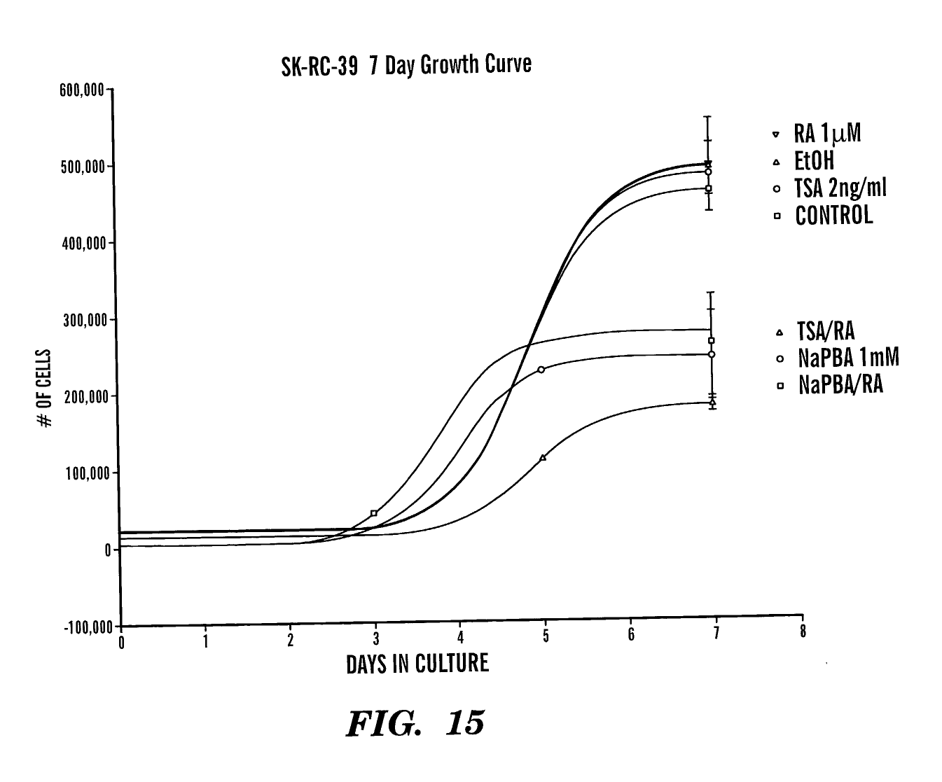

FIG. 15 is a growth curve of the kidney cancer line SK-RC-39, treated as indicated. The cell number was counted. The combination of retinoic acid plus low dose TSA was much more effective than either drug alone, as was the combination of phenylbutyrate plus retinoic acid, when compared to retinoic acid alone.

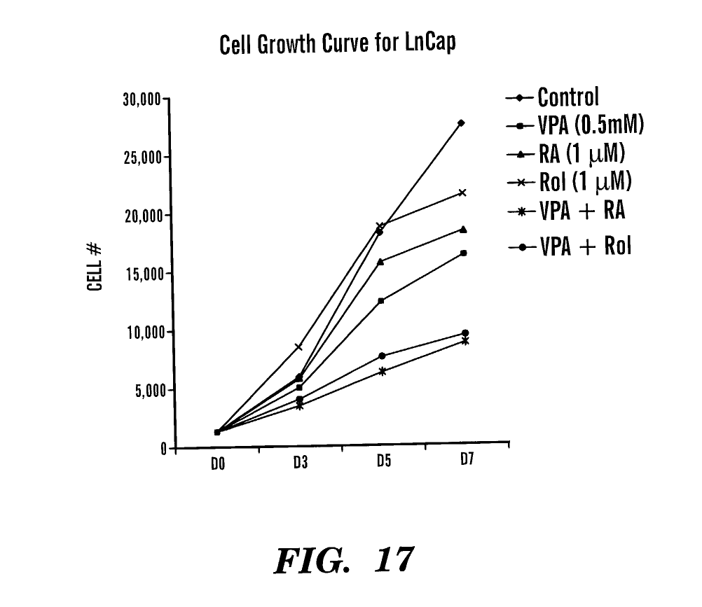

FIG. 17 graphically depicts the results of growth assays of the human prostate cancer cell line LnCap, treated as indicated. Cell number was counted on days 0, 3, 5, and 7. In this experiment, retinoic acid was employed alone at 1 μM. Retinol (vitamin A) was employed at 1 μM. Valproic acid, a histone deacetylase inhibitor, was employed at 0.5 μM alone. Combinations of these drugs were also used. All drugs were added only once, at time 0, in this experiment. As can be seen in FIG. 17, retinoic acid alone and retinol alone inhibited cell growth by 25% and approximately 27%, respectively, at day 7. Valproic acid (VPA) alone inhibited cell growth by approximately 40%. The combination of retinoic acid plus VPA inhibited cell growth by 75%, as did the combination of valproic acid plus retinol. Thus, the combination of the retinoids plus the histone deacetylase inhibitor VPA was strikingly more effective in inhibiting cell growth than each of these drugs at the same concentrations provided to the cells alone.

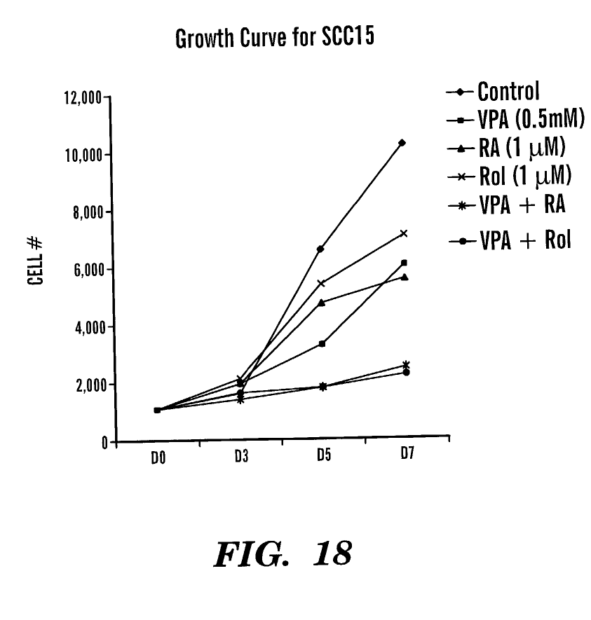

Results of growth assays of the human squamous cell carcinoma head and neck cell line SCC-15, are shown in FIG. 18. Cells were treated as indicated. Drugs were added only at time 0. Cell number was counted on days 0, 3, 5, and 7. As in FIG. 17, it can be seen that the combination of VPA plus retinoic acid or the combination of VPA plus retinol was much more effective in inhibiting cell growth than each of the drugs provided to the cells at the same concentrations alone.

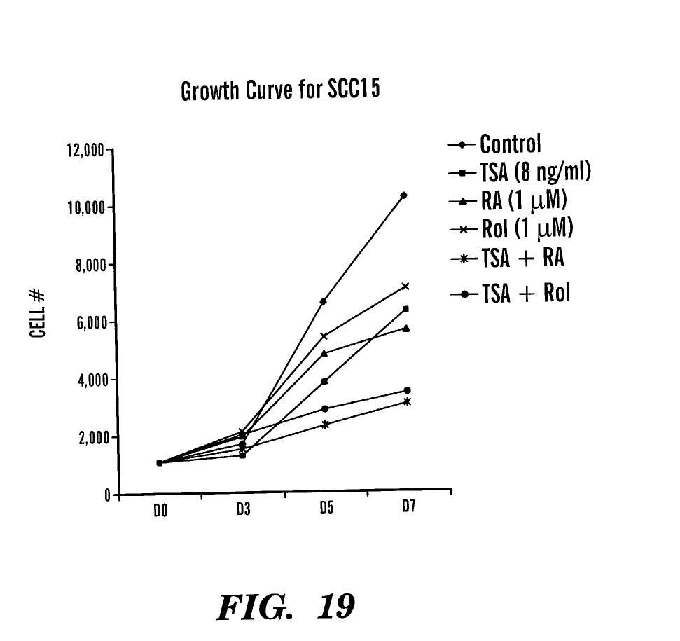

FIG. 19 graphically depicts the results of growth assays of the head and neck cancer cell line SCC-15, treated as indicated. Cell number was counted on days 0, 3, 5, and 7. In this experiment, a low dose of TSA (8 ng/ml) was used alone or in combination with 1 μM retinoic acid or 1 μM retinol. Drugs were added only at time 0. The TSA alone, retinoic acid alone, and retinol alone each inhibited cell growth at day 7 by approximately 35-40%. The combination of TSA plus retinoic acid or TSA plus retinol inhibited cell growth by almost 80%. Thus, the low dose histone deacetylase inhibitor combined with retinoic acid or retinol improved the effect of retinoic acid in reducing cell growth considerably.

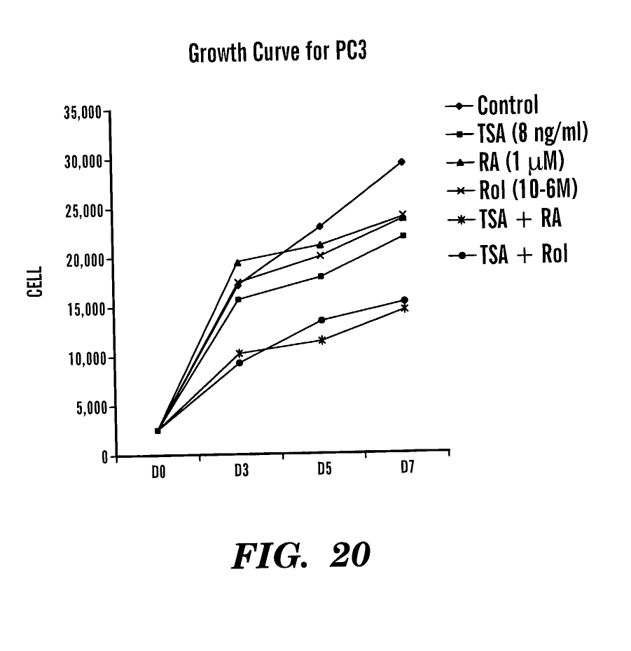

Results of growth assays of the human prostate cancer cell line PC-3, are depicted in FIG. 20. Cells were treated as indicated. Cell number was counted on days 0, 3, 5, and 7. Drugs were added only once on day 0. TSA alone, retinoic acid alone, and retinol alone each inhibited cell growth by approximately 20% at day 7. The combination of low dose TSA plus retinoic acid or low dose TSA plus retinol inhibited cell growth by almost 60%. Again, results indicate that the combination of a retinoid plus a histone deacetylase inhibitor resulted in more effective cell growth inhibition.

### EXAMPLE 2

### TUMOR XENOGRAFT MODEL

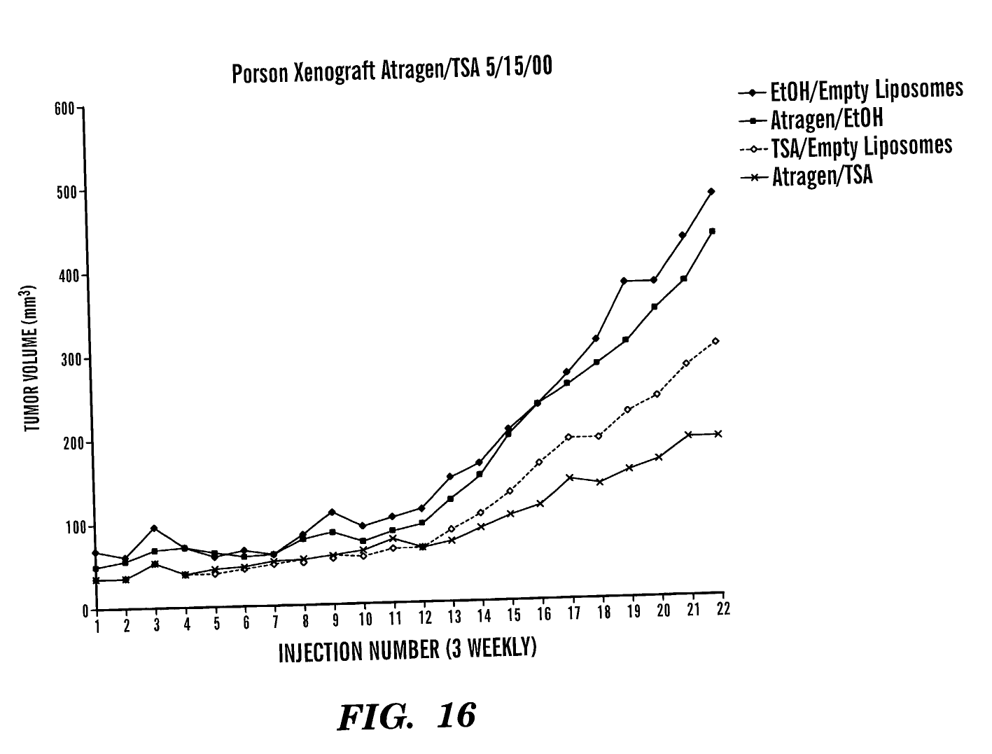

Since biologic effects were observed in cultured cells (FIGS. 1-15), and growth assays indicated synergy with the ATRA/TSA combination, tumor growth inhibition by treatment with combination ATRA/TSA therapy in a xenograft model was tested. Forty Swiss nu/nu mice were injected in the right flank subcutaneously with 5×10⁶ SK-RC-39 cells that had been cultured in 7% MEM and then trypsinized. Four days later, four cohorts of 10 mice each began to receive one tail vein injection of TSA or control and one injection of ATRA-IV or control every Monday, Wednesday and Friday for the duration of the experiment. The four treatment groups were as follows: (1) PBS with 1% EtOH+empty liposomes; (2) LipoATRA (7.4-8.1 mg/kg)+PBS with 1% EtOH; (3) TSA (34-41 μg/ml)+empty liposomes; and (4) TSA+LipoATRA. The study ended in the eighth week of treatment and at least six animals remained in each arm at the conclusion of the study and were considered evaluable. The animals tolerated the treatment well and gained weight throughout the treatment course. The tumor growth in the cohort that received LipoATRA +1% EtOH control was no different from that observed with the control arm. The tumor growth in the cohort that received TSA+empty liposomes was inhibited by 38% compared to control, while the inhibition of tumor growth seen in the TSA+LipoATRA arm was 61%. The tumor growth inhibition observed in the TSA+LipoATRA arm was much greater than that observed in the control arm. While the difference between the TSA+LipoATRA and the TSA+empty liposome arm was significant (FIG. 16), both of these treatments led to a very statistically significant difference in growth as compared to the control arm. Histologic analysis of the liver, lung, spleen and kidneys revealed no evidence of toxicity from either drug in these mice.

The foregoing results demonstrate the synergistic growth inhibition of human tumor cell lines by combination therapy with ATRA-IV and the HDAC inhibitor TSA, and the efficacy and lack of toxicity of TSA when given to a mouse tumor xenograft model. This is also the first report documenting the lack of toxicity of TSA in animals as well as in vivo effectiveness and the first report of in vivo synergy of ATRA with an HDAC inhibitor in a solid tumor malignancy.

What is claimed is:

1. A method of inhibiting growth of solid tumors in an animal which comprises administering an effective amount of trichostatin A (TSA) to an animal in need of such treatment.

2. A method of inhibiting growth of solid tumors in an animal which comprises administering an effective amount of a histone deacetylase inhibitor and a retinoid to an animal in need of such treatment.

3. The method of claim 1 or 2 wherein the solid tumor is selected from the group consisting of carcinomas of the head and neck, breast, skin, kidney, oral cavity, colon, prostate, pancreas or lung.

4. The method of claim 2 wherein the histone deacetylase inhibitor and retinoid are administered in combination.

5. The method of claim 2 wherein the histone deacetylase inhibitor and retinoid are administered serially.

6. The method of claim 2 wherein the histone deacetylase inhibitor is at least one of Trichostatin A, Trichostatin C, butyric acid, potassium butyrate, sodium butyrate, ammonium butyrate, lithium butyrate, phenylbutyrate, sodium phenylbutyrate (NaPBA), a stable butyrate derivative, traponin, valproic acid (VPA) or suberoylanilide hydroxamic acid (SAHA).

7. The method of claim 2 wherein the retinoid is at least one of retinol, 9-cis retinoic acid, 13-cis retinoic acid, all-trans retinoic acid, 4-oxoretinol, 4-oxoretinaldehyde or a retinyl ester.

8. The method of claim 7 wherein the retinoid is in a lipid formulation.

9. The method of claim 8 wherein the retinoid is a liposomal tretinoin such as liposomal ATRA Tretinoin or ATRA-IV.

10. The method of claim 1 or 2 wherein the animal is a mammal.

11. The method of claim 10 wherein the mammal is a human.
